(19) United States
(12) Patent Application Publication
(10) Pub. No.: US 2023/0285371 A1
(43) Pub. Date: Sep. 14, 2023

(54) USE OF SORAFENIB IN TREATMENT OF TYPE 1 DIABETES

(71) Applicant: THE SECOND XIANGYA HOSPITAL OF CENTRAL SOUTH UNIVERSITY, Changsha, Hunan (CN)

(72) Inventors: Tuo DENG, Changsha, Hunan (CN); Qin ZENG, Changsha, Hunan (CN); Jianfeng SONG, Changsha, Hunan (CN); Yang XIAO, Changsha, Hunan (CN); Zhiguang ZHOU, Changsha, Hunan (CN)

(73) Assignee: THE SECOND XIANGYA HOSPITAL OF CENTRAL SOUTH UNIVERSITY, Changsha, Hunan (CN)

(21) Appl. No.: 18/020,966

(22) PCT Filed: Jun. 28, 2021
(86) PCT No.: PCT/CN2021/102606

§ 371 Date: Feb. 13, 2023

(30) Foreign Application Priority Data
Aug. 13, 2020 (CN) 202010812329.3

(51) Int. Cl. A61K 31/44; A61P 3/10

(52) U.S. Cl. CPC A61K 31/44; A61P 3/10

(57) ABSTRACT

A use of Sorafenib or a derivative thereof in the preparation of a medication for the prevention and/or treatment of type 1 diabetes.

### CROSS REFERENCE TO RELATED APPLICATIONS

This application claims the priority of Chinese Patent Application No. 202010812329.3 filed with the China National Intellectual Property Administration on Aug. 13, 2020, and titled with “USE OF SORAFENIB IN TREATMENT OF TYPE 1 DIABETES”, which is hereby incorporated by reference.

### FIELD

The present disclosure relates to the field of medicine, and in particular to a new use of Sorafenib, especially to a use of Sorafenib in the preventive treatment of type 1 diabetes.

### BACKGROUND

Type 1 diabetes (T1D) is an autoimmune disease characterized by massive damage to the insulin-producing β cells of the pancreatic islet. It is extremely harmful and can cause a variety of complications, including cardiovascular disease, stroke, neurological disease, diabetic foot and so on. The incidence rate of type 1 diabetes is increasing year by year, and the incidence rate thereof in children worldwide is doubling every 20 years. At present, the treatment of type 1 diabetes mainly relies on insulin, but long-term injection of insulin not only brings inconvenience to patients, but also causes many complications. Therefore, it is urgent to find a new treatment for type 1 diabetes.

The destruction of β cells in T1D is mainly mediated by autoreactive T cells, so targeting autoreactive T cells brings new hope for the treatment of T1D. Currently, a variety of T cell-based immunotherapies have emerged, such as anti-CD3 monoclonal antibodies, CTLA4-Ig fusion proteins, and low-dose antithymocyte globulin. These immunotherapies do show some efficacy in human clinical trials, but their systemic immunosuppressive effects and transient effectiveness limit their clinical application. Facing the growing number of T1D populations, there is an urgent need to develop more effective and safer T cell-based immunotherapies.

In the early stages of T1D, antigen-presenting cells (APCs) present self-antigens such as precursor pro-insulin (PPI), insulinoma-associated antigen 2 (I-A2), glutamate decarboxylase (GAD), zinc transporter (ZnT8) and the like to CD4+ T cells. Meanwhile, APC secretes interleukin 12 (IL-12) to induce naive CD4+ T cells to differentiate into Th1 cells. Th1 cells produce inflammatory cytokines including IFN-γ and IL-2 to activate CD8+ cytotoxic T lymphocytes and macrophages, and cooperate with them to attack pancreatic β cells. Blocking Th1 cell differentiation by disrupting Tbet, a key transcription factor for Th1 differentiation, can effectively block the occurrence of insulitis and T1D in NOD mice. Thus, the Th1 cell population is a key mediator of T1D pathogenesis. IL-12 is a key cytokine that induces Th1 cell differentiation, and the IL-12-induced activation of signal transducer and transcription 4 (Stat4) is an important signaling pathway driving Th1 cell differentiation. Accumulating evidence suggests that the differentiation of Th1 cells induced by IL-12 plays a crucial role in the pathogenesis of T1D. In NOD mice, daily injection of IL-12 may increase the incidence rate of T1D, whereas administration of IL-12 antagonists may decrease the incidence rate of T1D. Furthermore, inhibition of Stat4 activation can completely prevent the development of T1D in NOD mice. In humans, the IL-12b gene is considered a T1D-related gene. In sum, these studies suggest that the IL-12-induced Th1 differentiation plays a critical role in the pathogenesis of T1D. Therefore, targeting the IL-12/Stat4 axis to inhibit Th1 cell differentiation may be a more specific and effective method for the treatment of T1D.

Tyrosine kinases are a group of enzymes with catalytic subunits that can transfer phosphate groups on ATP to one or more tyrosine residues in proteins, resulting in conformational changes of proteins that affect protein function. Tyrosine kinases are important mediators of signal transduction, which can regulate cellular reactions such as cell division, differentiation, apoptosis and metabolism in response to external and internal stimuli. Since tyrosine kinases are involved in a variety of biological processes, tyrosine kinase inhibitors (TKIs) have been thus developed for the treatment of various diseases, including cancer, infectious diseases, autoimmune diseases, inflammatory diseases, etc. To date, 39 TKIs have been approved by the FDA, and more TKIs are being tested in clinical trials. The IL-12/Stat4 pathway is directly mediated by two tyrosine kinases, Janus kinase 2 (Jak2) and tyrosine kinase 2 (Tyk2), and may be indirectly regulated by other tyrosine kinases.

It is of important practical significance to discover drugs that can inhibit differentiation of Th1 cells induced by IL-12 and have a therapeutic effect on T1D.

### SUMMARY

In view of this, the present invention provides use of Sorafenib in the preparation of an oral medicament for the preventive treatment of type 1 diabetes. Sorafenib for the preventive treatment of type 1 diabetes provides a new treatment approach for the majority of patients, and provides more choices for clinicians and patients. For patients with type 1 diabetes, Sorafenib provides an oral medicament without the hassle of daily insulin injections.

In order to achieve the above-mentioned purpose of the present invention, the present invention provides the following technical solutions:

In view of this, the present invention provides use of Sorafenib in the preparation of an oral medicament for the preventive treatment of type 1 diabetes. Sorafenib for the preventive treatment of type 1 diabetes provides a new treatment approach for the majority of patients, and provides more choices for clinicians and patients. For patients with type 1 diabetes, Sorafenib provides an oral medicament without the hassle of daily insulin injections.

In order to achieve the above-mentioned purpose of the present invention, the present invention provides the following technical solutions:

Sorafenib is a small molecule compound that inhibits tumor cell proliferation and angiogenesis and increases apoptosis in a wide range of tumor models. As an oral receptor tyrosine kinase inhibitor (TKI), it inhibits factors involved in tumorigenesis and tumor progression, such as Raf serine/threonine kinase and receptor tyrosine kinase (vascular endothelial growth factor receptor 1, 2, 3 and platelet-derived growth factor-β, Flt-3 and c-kit). Sorafenib has been approved by the US FDA for the treatment of advanced inoperable hepatocellular carcinoma (HCC), advanced renal cell carcinoma (RCC), and advanced radioiodine-refractory differentiated thyroid cancer (RRDTC). Sorafenib has a molecular formula of C₂₁H₁₆ClF₃N₄O₃, a molecular weight of 464.82500, and a structural formula as shown in formula I.

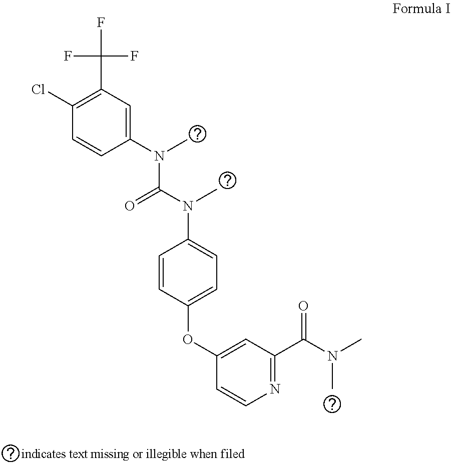 [SMILES: CN(C)C(=O)c1cc(Oc2ccc(N(C)C(=O)N(C)c3ccc(Cl)c(C(F)(F)F)c3)cc2)ccn1]

In the present invention, the preventive and therapeutic effects of Sorafenib on type 1 diabetes were clarified by cell and mouse models.

In some embodiments of the present invention, Naive CD4+ T cells cultured in vitro were first treated with increasing concentrations of Sorafenib, then induced with IL-12 to differentiate into Th1 cells, and the proportion of Th1 was detected by flow cytometry. The results showed that Sorafenib inhibits differentiation of Th1 cells induced by IL-12 in a dose-dependent manner.

In some embodiments of the present invention, Naive CD4+ T cells were treated with increasing concentrations of Sorafenib for two days, and the treated cells were counted by a cytometer. The results showed that Sorafenib is not cytotoxic to T cells.

It can be seen that Sorafenib can be used to inhibit the differentiation of Th1 cells induced by IL-12.

In some embodiments of the present invention, female NOD mice were gavaged with Sorafenib before the onset of type 1 diabetes, and spontaneous incidence rate and cyclophosphamide-induced incidence rate in mice were observed. The results showed that Sorafenib can prevent the occurrence of type 1 diabetes in NOD mice.

It can be seen that Sorafenib can be used to prevent the occurrence of type 1 diabetes.

In some embodiments of the present invention, the mice were continuously gavaged with Sorafenib for 4 weeks before the onset of disease, then the nondiabetic mice were sacrificed, and the pancreas thereof was taken, fixed with formaldehyde, sliced, and stained with HE to observe the islet under a microscope, through which the insulitis in the Sorafenib group and the solvent group was compared. The results showed that the insulitis in the Sorafenib group was significantly improved compared with the solvent group, and the mononuclear cell infiltration thereof was significantly reduced.

In some embodiments of the present invention, the mice were continuously gavaged with Sorafenib for 4 weeks before the onset of disease, and injected with cyclophosphamide 2 weeks after the gavage treatment to induce the onset of disease, then the mice were sacrificed, and the pancreas thereof was taken, a small portion of which was used to extract RNA for RT-PCR to detect the gene expression levels of Th1 cell marker genes (Tbet and IFNγ) and pro-inflammatory cytokines (IL-1β, IL-6 and TNF-α) in the pancreas, and most of the rest of which was subjected to flow cytometry to detect the changes of CD4+ T cell subsets (Th1, Th2, Th17 and Treg) in the pancreas of the two groups of mice. The results showed that Sorafenib can reduce the infiltration of Th1 cells in pancreas and the expression of inflammatory factors.

It can be seen that Sorafenib can be used to protect the pancreas of NOD mice from the attack of autoreactive T cells.

Further, in some embodiments of the present invention, the Naive CD4+ T cells cultured in vitro were first treated with increasing concentrations of Sorafenib, then collected after 48 hours to extract proteins for western blot (WB) to detect the protein levels of STAT4 and p-STAT4. The results showed that Sorafenib is able to inhibit the phosphorylation of STAT4, and the dose of Sorafenib to inhibit STAT4 activation completely matches the dose thereof to inhibit Th1 differentiation.

It can be seen that Sorafenib inhibits differentiation of Th1 cells induced by IL-12 by inhibiting the activation of STAT4.

In conclusion, the present invention provides use of Sorafenib in the preventive treatment of type 1 diabetes.

Among them, the medicament is Sorafenib.

Further, the medicament also comprises a pharmaceutically acceptable excipient.

The medicament can be in any dosage form in the current pharmaceutical field, including oral preparations or injection preparations.

Each pharmaceutical dosage form can be prepared by selecting appropriate acceptable excipients according to the actual needs of the dosage form, which belongs to the conventional dosage form preparation technology in the art, such as capsules, tablets, injection powder and so on.

It can be known from the above technical solutions that the present invention provides the use of Sorafenib in the preventive treatment of type 1 diabetes. Sorafenib for the preventive treatment of type 1 diabetes provides a new treatment approach for the majority of patients, and provides more choices for clinicians and patients. For patients with type 1 diabetes, Sorafenib can provide an oral medicament therapy without the hassle of daily insulin injections. Sorafenib can be chemically synthesized, and the cost thereof is lower than that of biological preparations. In addition, it has been approved by FDA and NMDA for marketing and clinical treatment. It has few and mild side effects, which is well tolerated by clinical patients, and brings little burden on patients.

### BRIEF DESCRIPTION OF DRAWINGS

In order to illustrate the embodiments of the present invention or the technical solutions in the prior art more clearly, the following briefly introduces the drawings that are required in the description of the embodiments or the prior art.

FIG. 1 shows that in Example 1, Sorafenib inhibits the differentiation of Th1 cells induced by IL-12 in a dose-dependent manner; wherein, a shows the proportion of Th1 cells under treatment of different concentrations of Sorafenib detected by flow cytometry; b shows the quantitative analysis result;

FIG. 2 shows that in Example 2, Sorafenib has no cytotoxicity to T cells;

FIG. 3 shows that in Example 3, Sorafenib can prevent the occurrence of type 1 diabetes in NOD mice; wherein, a is the result diagram of Sorafenib preventing spontaneous type 1 diabetes in NOD mice; b is the result diagram of Sorafenib preventing cyclophosphamide-induced type 1 diabetes in NOD mice;

FIG. 4 shows that in Example 4, Sorafenib can improve the insulitis in NOD mice;

FIG. 5 shows that in Example 5, Sorafenib reduces the infiltration of Th1 cells in the pancreas of NOD mice; wherein a shows the changes of CD4+ T cell subsets (Th1, Th2, Th17 and Treg) in the pancreas of the two groups of mice detected by flow cytometry; b shows the gene expression levels of Th1 cell marker genes (Tbet and IFNγ) and pro-inflammatory cytokines (IL-1β, IL-6 and TNF-α) in the pancreas detected by RT-PCR;

FIG. 6 shows that in Example 6, Sorafenib inhibits differentiation of Th1 cells induced by IL-12 by inhibiting the phosphorylation of Stat4; wherein, a is the western blot result, and b is the quantitative result.

### DETAILED DESCRIPTION

The present invention discloses a new use of Sorafenib, and those skilled in the art can learn from the content of this document and appropriately improve the process parameters to achieve the present invention. It should be particularly noted that all similar replacements and modifications are obvious to those skilled in the art, and they are deemed to be included in the present invention. The method and use of the present invention have been described through the preferred embodiments, and it is obvious that those skilled in the art can make modifications or appropriate changes and combinations to the method and use described herein without departing from the content, spirit and scope of the present invention to achieve and apply the technology of the present invention.

Unless otherwise specified, the reagents involved in the examples of the present invention are all commercially available products, which can be purchased through commercial channels.

The present invention will be further elaborated below in conjunction with examples:

### Example 1. Sorafenib Inhibits Differentiation of Th1 Cells Induced by IL-12 in a Dose-Dependent Manner

I. Materials and Methods

1. Cells

C57BL/6J mice were sacrificed, the spleen was taken and disrupted in PBS containing 2% fetal bovine serum (FBS). The cell suspension was filtered through a 70 μm nylon filter to remove tissue debris. The obtained cell suspension was centrifuged at 300 g for 10 min, and the cells were resuspended in PBS containing 2% fetal bovine serum (FBS) to maintain a cell concentration of 1×10\hat{ }8 nucleated cells/mL. Naive CD4+ T cells were extracted with the EasySep™ Mouse Naive CD4+ T Cell Isolation Kit (STEMCELL Technologies), and the extracted Naive CD4+ T cells were cultured in RPMI-1640 medium (Gibco) containing 10% fetal bovine serum (Gibco) and 1% penicillin/streptomycin (Gibco).

2. Stimulatory Factors

IL-12 was purchased from peprotech, and Sorafenib was Nexavar tablets purchased from hospital. Both of IL-12 and Sorafenib were dissolved in DMSO to prepare stock solutions at a concentration of 10 mM, and the stock solutions were frozen at −80° C., which were diluted with RPMI medium as working solutions to treat cells.

3. In Vitro Inhibition Assay

In order to test the inhibitory effect of Sorafenib on differentiation of Th1 cells induced by IL-12, the above Naive CD4+ T cells were evenly seeded in 12-well plates, added with increasing concentrations of Sorafenib (concentration gradient: 0, 1, 2, 4 μg/ml), then supplemented with DMSO to an equal volume, 3 parallel replicate groups were set, and each well was added with an equal amount of IL-12. The cells were collected after 48 hours, and subjected to flow cytometry to detect the proportion of Th1 cells.

II. Analysis of the Results

In FIG. 1, a shows the proportion of Th1 cells under treatment of different concentrations of Sorafenib detected by flow cytometry, and b shows the quantitative analysis result. It can be seen that with the increase of Sorafenib concentration, the proportion of Th1 cells gradually decreased, suggesting that Sorafenib can inhibit IL-12-induced Th1 differentiation in a dose-dependent manner.

TABLE 1
FIG. 1b data

| | Sorafenib concentration (μg/ml) | | | |
|---|---|---|---|---|
| | 0 | 1 | 2 | 4 |
| Th1 (%) | 82.3 | 77.9 | 67.8 | 24.9 |
| | 82.4 | 76 | 68.5 | 12.9 |
| | 91.1 | 87.4 | 69.3 | 9.17 |
| Average | 85.27 | 80.43 | 68.53** | 15.66*** |

Three parallel wells were set in each group, different concentration groups were compared with 0 μg/ml respectively, and independent samples t test was used for statistical analysis: ** represents P value<0.01, *** represents P value<0.001.

### Example 2. Sorafenib is not Cytotoxic to T Cells

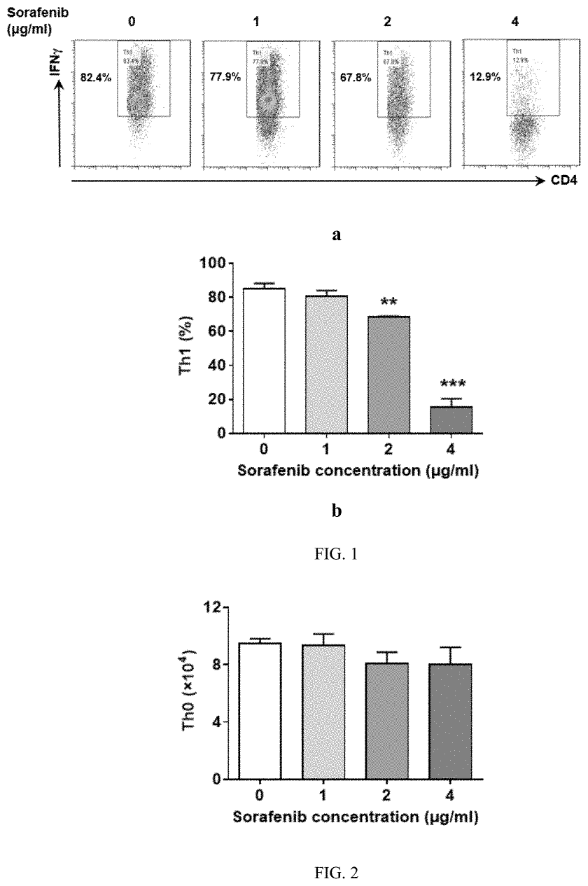

Method: Naive CD4+ T cells were treated with increasing concentrations of Sorafenib, and after 48 hours the treated cells were collected and counted. The results are shown in FIG. 2.

The results show that Sorafenib treatment did not affect the number of Naive CD4+ T cells, and it was not cytotoxic to Naive CD4+ T cells.

TABLE 2
FIG. 2 data

| | Sorafenib concentration (μg/ml) | | | |
|---|---|---|---|---|
| | 0 | 1 | 2 | 4 |
| Th0 (×10\hat{ }4) | 10 | 9.2 | 9.6 | 4.8 |
| | 9.6 | 8 | 8 | 7.6 |
| | 8.8 | 10.8 | 6 | 10.4 |
| | | | 8.8 | 9.2 |
| Average | 9.47 | 9.33 | 8.1 | 8 |

### Example 3. Sorafenib can Prevent the Occurrence of Type 1 Diabetes in NOD Mice

I. Materials and Methods
1. Mice: The most widely used spontaneous type 1 diabetes mouse model for the study of type 1 diabetes—NOD mice were selected. Since the incidence rate of spontaneous type 1 diabetes in female NOD mice is much higher than that in male mice, female NOD mice were used for experiments in the present invention. The female NOD mice of the present invention were purchased from Shanghai Model Organisms Center, Inc.
2. Preparation of medicine: Carboxymethylcellulose (CMC) was used as the solvent. Before preparation of medicine, CMC was autoclaved, the tablet Sorafenib was ground into powder on an ultra-clean workbench, then an appropriate amount of Sorafenib powder was weighed so as to be uniformly suspended in the solvent, and the prepared medicine was stored at 4° C.
3. Administration: In the present invention, the medicine was intragastrically administered to the mice, and the administration concentration of Sorafenib was 10 mg/kg body weight.
4. Injection of cyclophosphamide: 300 mg/kg body weight of cyclophosphamide was injected into NOD mice so as to rapidly induce the onset of type 1 diabetes.
5. Measurement of blood sugar: About 1 mm of the tail tip of the mice was cut off, the first drop of blood was wiped off with gauze, then the second drop of blood flew out naturally, which was drawn with a blood sugar test strip, and the reading was recorded. It can be diagnosed type 1 diabetes when two consecutive blood sugar readings were both >250 mg/dl.
6. Observation of the preventive effect of Sorafenib on spontaneous type 1 diabetes in NOD mice: the mice were divided into two groups, one group was the Sorafenib-gavaged group, and the other was the CMC-gavaged group. From the age of 8 weeks, the mice were gavaged once a day for 12 consecutive weeks, and the spontaneous incidence rate of type 1 diabetes in the mice was observed and counted.
7. Observation of the preventive effect of Sorafenib on the occurrence of type 1 diabetes induced by cyclophosphamide in NOD mice: the mice were divided into two groups, one was the Sorafenib-gavaged group, and the other was the CMC-gavaged group. From the age of 8 weeks, the mice were gavaged once a day for 7 consecutive weeks. After 4 weeks of gavage, each mouse was injected with cyclophosphamide to induce the onset of type 1 diabetes, and the incidence rate of type 1 diabetes in the mice was observed and counted.

II. Analysis of the Results

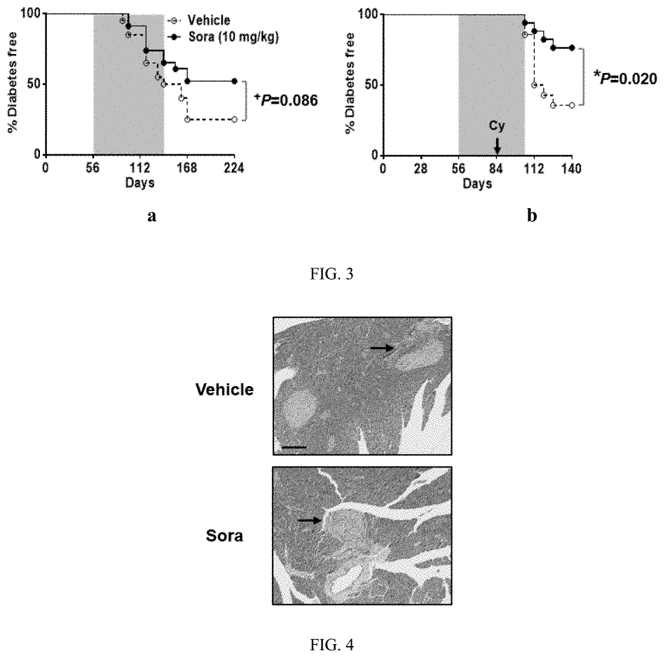

In FIG. 3, a shows the preventive effect of Sorafenib on spontaneous type 1 diabetes in NOD mice. It was observed that at 32 weeks of age, the incidence rate of type 1 diabetes in the mice of Sorafenib group was 48%, while the incidence rate in the mice of CMC group was 75%. b shows the preventive effect of Sorafenib on the occurrence of type 1 diabetes induced by cyclophosphamide in NOD mice. Cyclophosphamide injection caused rapid onset of disease in NOD mice, and more than half of the CMC group developed type 1 diabetes, while the incidence rate in the Sorafenib group was only 24%. The preventive effect on type 1 diabetes in the Sorafenib group still existed after the gavage was stopped, and lasted to 20 weeks, suggesting that Sorafenib has preventive treatment effect on type 1 diabetes.

TABLE 3
FIG. 3a data

| | Survival rate (%) | |
|---|---|---|
| Days | Vehicle | Sora |
| 0 | 100 | 100 |
| 91 | 95 | |
| 98 | 85 | 91.30 |
| 119 | 65 | 73.91 |
| 133 | 55 | |
| 140 | 50 | 65.22 |
| 154 | | 60.87 |
| 161 | 40 | |
| 168 | 25 | 52.17 |
| 224 | 25 | 52.17⁺ |

There were 20 mice in the CMC-gavaged (vehicle) group and 23 mice in the Sorafenib-gavaged (sora) group. The two groups of mice were subjected to survival analysis at different time points: ⁺ represents P value <0.1.

TABLE 4
FIG. 3b data

| | Survival rate (%) | |
|---|---|---|
| Days | Vehicle | Sora |
| 0 | 100 | 100 |
| 105 | 85.71 | 94.12 |
| 112 | 50 | 88.24 |
| 119 | 42.86 | 82.35 |
| 126 | 35.71 | 76.47 |
| 140 | 35.71 | 76.47* |

There were 14 mice in the CMC-gavaged (vehicle) group and 17 mice in the Sorafenib-gavaged (sora) group. The two groups of mice were subjected to survival analysis at different time points: * represents P value <0.05.

### Example 4. Sorafenib can Improve Insulitis in NOD Mice

Methods: NOD mice were divided into two groups, one group was Sorafenib-gavaged group, and the other was CMC-gavaged group. From the age of 8 weeks, the mice were gavaged once a day for 4 consecutive weeks. The nondiabetic mice were sacrificed, and the pancreas thereof was taken out, fixed with formaldehyde, sliced, stained with HE to observe the islet under a microscope, and photographed.

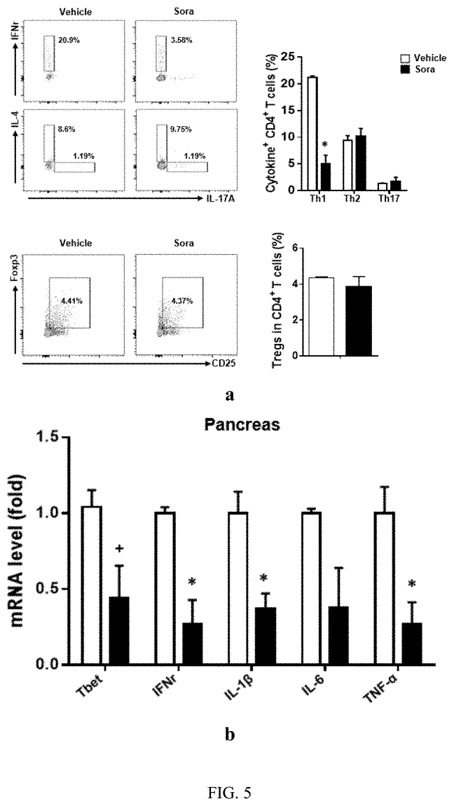

The results in FIG. 4 show that, compared with the CMC group, the insulitis in the Sorafenib group was significantly improved, and the infiltration of mononuclear cells therein was significantly reduced.

### Example 5. Sorafenib Reduces the Infiltration of Th1 Cells in Pancreas of NOD Mice

Methods: The mice were divided into two groups, one group was the Sorafenib-gavaged group, the other was the solvent-gavaged group. From the age of 10 weeks, the mice were gavaged for 4 weeks, and injected with cyclophosphamide after the gavage for two weeks to induce the onset of the disease. Then the mice were sacrificed, and the pancreas thereof was taken, a small portion of which was used to extract RNA for RT-PCR, and most of the rest of which was subjected to flow cytometry.

Results: In FIG. 5, a shows the changes of CD4+ T cell subsets (Th1, Th2, Th17 and Treg) in the pancreas of the two groups of mice detected by flow cytometry. The results show that the proportion of Th1 cells in the pancreas of the mice in Sorafenib group was significantly reduced, while the proportions of other T cell subsets remained unchanged; b shows the gene expression levels of Th1 cell marker genes (Tbet and IFNγ) and pro-inflammatory cytokines (IL-1β, IL-6 and TNF-α) in the pancreas detected by RT-PCR. The results show that the gene expression levels of Th1 cell marker genes (Tbet and IFNγ) and pro-inflammatory cytokines (IL-1β, IL-6 and TNF-α) in the pancreas of the Sorafenib group were significantly reduced, suggesting that Sorafenib prevents the development of type 1 diabetes by reducing the infiltration of pancreatic Th1 cells in NOD mice.

TABLE 5
FIG. 5a data

| | Vehicle | | Average | Sora | | | Average |
|---|---|---|---|---|---|---|---|
| Th1 (%) | 21.5 | 20.9 | 21.2 | 6.63 | 3.58 | | 5.11* |
| Th2 (%) | 8.6 | 10.3 | 9.45 | 9.75 | 12.9 | 7.91 | 10.19 |
| Th17 (%) | 1.43 | 1.19 | 1.31 | 3.28 | 0.68 | 1.19 | 1.72 |

Two or three parallel wells were set in each group, the sora group was compared with the vehicle group, and the independent samples t-test was used for statistical analysis: * represents P value <0.05.

TABLE 6
FIG. 5a data

| | Vehicle | | Average | Sora | | | | Average |
|---|---|---|---|---|---|---|---|---|
| Tregs (%) | 4.41 | 4.28 | 4.345 | 4.6 | 4.31 | 2.25 | 4.37 | 3.88 |

TABLE 7
FIG. 5b data

| | Vehicle | | | Average | Sora | | | Average |
|---|---|---|---|---|---|---|---|---|
| Tbet | 1 | 1.25 | 0.87 | 1.04 | 0.27 | 0.2 | 0.86 | 0.44⁺ |
| IFNγ | 0.96 | 1.04 | | 1 | 0.16 | 0.07 | 0.58 | 0.27* |
| Il-1β | 0.9 | 0.82 | 1.28 | 1 | 0.48 | 0.18 | 0.46 | 0.37* |
| IL-6 | 0.97 | 1.03 | | 1 | 0.64 | 0.12 | | 0.38 |
| TNFα | 1 | 0.7 | 1.3 | 1 | 0.18 | 0.07 | 0.55 | 0.27* |

There were 2 or 3 mice in each group, the sora group was compared with the vehicle group, and the independent samples t-test was used for statistical analysis: ⁺ represents P value <0.1, * represents P value <0.05.

### Example 6. Sorafenib Inhibits Differentiation of Th1 Cells Induced by IL-12 by Inhibiting the Phosphorylation of Stat4

Method: Naive CD4+ T cells were treated with increasing concentrations of Sorafenib (concentration gradient: 0, 1, 2, 4 μg/ml), then supplemented with DMSO to an equal volume, 3 parallel replicate groups were set, and each well was added with an equal amount of IL-12. The cells were collected after 48 hours to extract proteins for western blot (WB) to detect the protein levels of STAT4 and p-STAT4.

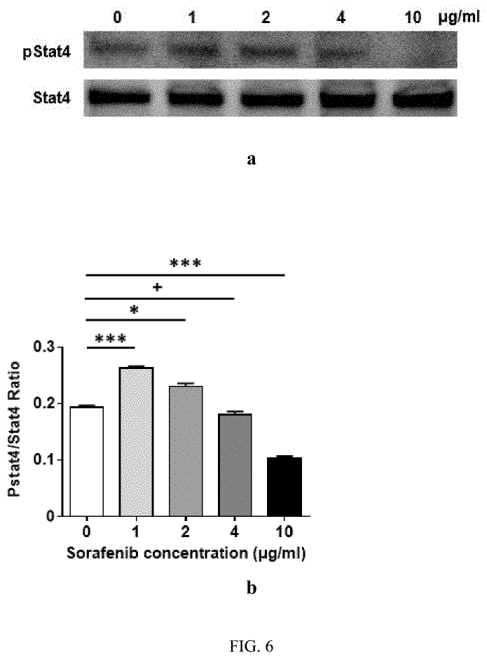

Results: In FIG. 6, a is the WB result, and b is the quantitative result. The results show that Sorafenib can inhibit the phosphorylation of STAT4, and the dose of Sorafenib to inhibit STAT4 activation completely matches the dose thereof to inhibit Th1 differentiation, which shows that Sorafenib inhibits differentiation of Th1 cells induced by IL-12 by inhibiting the activation of STAT4.

TABLE 8
FIG. 6b data

| | Sorafenib concentration (μg/ml) | | | | |
|---|---|---|---|---|---|
| | 0 | 1 | 2 | 4 | 10 |
| p-stat4/Stat4 Ratio | 0.2 | 0.27 | 0.24 | 0.19 | 0.11 |
| | 0.19 | 0.26 | 0.23 | 0.17 | 0.1 |
| | 0.19 | 0.26 | 0.22 | 0.18 | 0.1 |
| Average | 0.19 | 0.26*** | 0.23* | 0.18⁺ | 0.10*** |

Three parallel wells were set in each group, the different concentration groups were compared with 0 μg/ml respectively, and the independent sample t test was used for statistical analysis: ⁺ represents P value <0.1, * represents P value <0.05, ** represents P value <0.01, and *** represents P value <0.001.

The new use of Sorafenib provided by the present invention, especially the use of Sorafenib in the preventive treatment of type 1 diabetes, has been introduced in detail above. The principles and implementations of the present invention are described herein by using specific examples, and the descriptions of the above examples are only used to help understand the method and the core idea of the present invention. It should be noted that for those skilled in the art, several improvements and modifications can also be made to the present invention without departing from the principle of the present invention, and these improvements and modifications also fall within the protection scope of the claims of the present invention.

1. (canceled)

2. A method of preventing and/or treating type 1 diabetes, comprising administering Sorafenib or a derivative thereof to a subject in need thereof.

3. A method of improving pancreatic islet function in type 1 diabetes, comprising administering Sorafenib or a derivative thereof to a subject in need thereof.

4. The method according to claim 3, wherein improving pancreatic islet function in type 1 diabetes is to improve insulitis or reduce infiltration of mononuclear cells.

5. (canceled)

6. A method of inhibiting STAT4 activation, comprising administering Sorafenib or a derivative thereof to a subject in need thereof.

7. The method according to claim 2, wherein Sorafenib or a derivative thereof is administered in the form of a medicament, and the medicament comprises Sorafenib or a derivative thereof a pharmaceutically acceptable excipient.

8. The method according to claim 2, wherein Sorafenib or a derivative thereof is administered in the form of a medicament, and the medicament is an oral preparation or an injection preparation.
